(19) United States
(12) Patent Application Publication
(10) Pub. No.: US 2020/0405352 A1
(43) Pub. Date: Dec. 31, 2020

(54) METHODS FOR TRANSCERVICAL STERILIZATION

(71) Applicant: Covidien LP, Mansfield, MA (US)

(72) Inventors: Nikolai D. Begg, Wellesley, MA (US); Chad A. Pickering, Woburn, MA (US); Jordan A. Whisler, Brookline, MA (US); Lisa M. Quealy, Dracut, MA (US); Jacob J. Kelly, North Easton, MA (US)

(21) Appl. No.: 16/911,450

(22) Filed: Jun. 25, 2020

Related U.S. Application Data
(60) Provisional application No. 62/867,555, filed on Jun. 27, 2019.

(51) Int. Cl. A61B 17/42; A61B 17/3211; A61B 34/35

(52) U.S. Cl. CPC A61B 17/42; A61B 2017/0647; A61B 34/35; A61B 17/3211

(57) ABSTRACT

A method of sterilizing a female patient includes cutting into a fundus of a uterus of the female patient to form a flap. The method also includes securing the flap to a wall of the uterus to occlude a fallopian tube of the female patient.

### CROSS REFERENCE TO RELATED APPLICATIONS

This application claims the benefit of U.S. Provisional Application Ser. No. 62/867,555, filed Jun. 27, 2019, the entire contents of which are incorporated by reference herein.

### TECHNICAL FIELD

This disclosure relates to surgical methods, and more particularly, to methods for occluding fallopian tubes to effectuate sterilization.

### BACKGROUND

Female sterilization permanently prevents women from becoming pregnant. Known procedures include tubal ligation and tubal implants. They both work by blocking the fallopian tubes so that sperm cannot meet with and fertilize an egg. In tubal ligation procedures, also known as “having your tubes tied,” the fallopian tubes are cut, sealed, clipped, or tied to prevent pregnancy immediately, but requires several incisions to be made in the abdomen. For tubal implant procedures, a very small spring-like coil is placed into each fallopian tube. The coils cause scar tissue to form in the tubes, blocking the tubes. This method does not involve cuts or incisions. Instead, a clinician uses a thin tube to thread the small coils through the vagina and uterus into the fallopian tubes, where the coils will remain so that scar tissue can build up around the coils to occlude the tubes. It can take up to three months for the scar tissue to fully occlude the tubes.

### SUMMARY

In accordance with an aspect of this disclosure, a method of sterilizing a female patient includes cutting into a fundus of a uterus of the female patient to form a flap. The method further includes securing the flap to a wall of the uterus to occlude a fallopian tube of the female patient.

In aspects, the method further involves forming the flap adjacent to a tubal ostia of the fallopian tube. The method may comprise forming a second flap from the fundus of the uterus. The method may involve positioning the second flap adjacent to a tubal ostia of a second fallopian tube of the female patient.

In aspects, the method includes securing the second flap to the wall of the uterus to occlude the second fallopian tube. Securing the second flap may include fastening the second flap to the body with a tack.

In aspects, securing the flap may include fastening the flap to the body with a tack.

In aspects, the method may involve advancing a cutter into the uterus transcervically. Cutting into the fundus may include conducting electrical energy through the cutter.

In aspects, forming the flap may include positioning the flap to depend from the fundus.

The details of one or more aspects of this disclosure are set forth in the accompanying drawings and the description below. Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a front view illustrating female reproductive anatomy with a surgical instrument inserted into a patient's uterus in accordance with the principles of this disclosure; and

FIGS. 2 and 3 are progressive views illustrating a method for transcervical sterilization of the female reproductive anatomy with the surgical instrument of FIG. 1.

### DETAILED DESCRIPTION

Aspects of this disclosure are described in detail with reference to the drawings, in which like reference numerals designate identical or corresponding elements in each of the several views. As commonly known, the term “clinician” refers to a doctor (e.g., a surgeon), a nurse, or any other care provider and may include support personnel. In the following description, well-known functions or constructions are not described in detail to avoid obscuring this disclosure in unnecessary detail.

In general, this disclosure describes a method for occluding fallopian tubes to effectuate immediate sterilization without abdominal incisions.

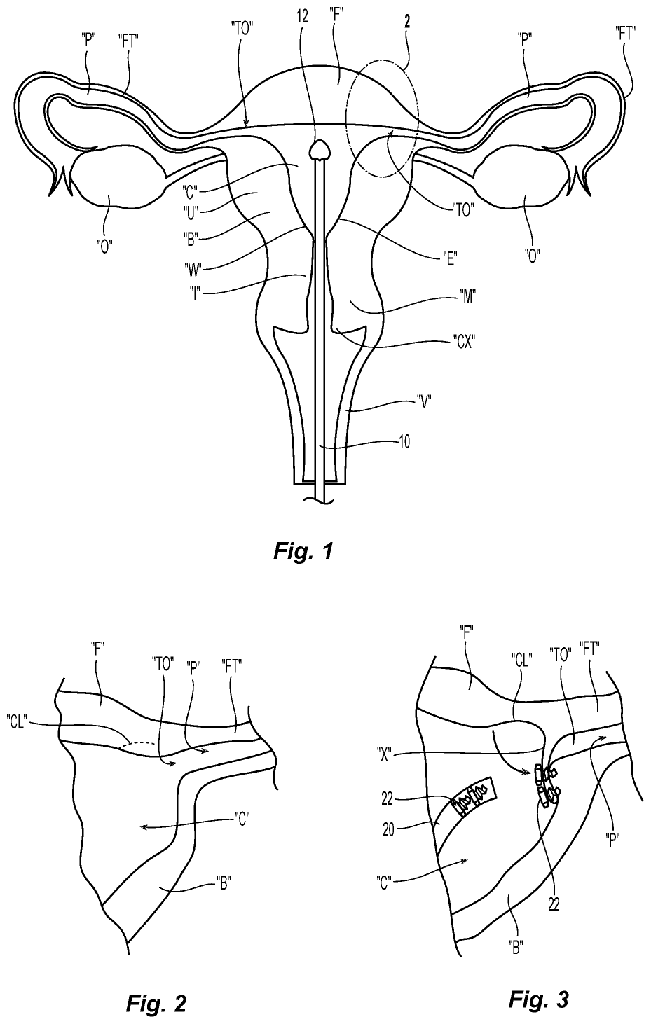

As seen in FIGS. 1-3, the female reproductive organs include the vagina “V”, the uterus “U,” the fallopian tubes “FT,” and the ovaries “0.” There are two fallopian tubes “FT” attached to a side of the uterus “U” that open into the uterus “U” at tubal ostia “TO.” Each fallopian tube “FT” defines a passageway “P” that leads to one of the ovaries “0.” The uterus “U” includes the fundus “F” or the broad curved upper area to which the fallopian tubes “FT” attach. The uterus “U” also includes the body “B” or the main part of the uterus “U” which starts directly below the fallopian tubes “FT” and continues downward until walls “W” of the body “B” and the uterine cavity “C” defined by the walls “W” of the body “B” begin to narrow. The isthmus “I” of the uterus “U” is the lower, narrow neck region of the uterus “U” that extends to the cervix “CX,” which opens into the vagina “V.” Lining the uterine cavity “C” is a mucous membrane known as the endometrium “E” under which the myometrium “M” or the smooth muscle tissue of the uterus “U,” is disposed.

To sterilize a female patient, a clinician can insert (e.g., transvaginally) a surgical instrument 10, for example, an electrosurgical instrument with a cutter 12 such as a loop electrode for transecting and/or resecting tissue, into the uterus “U” (e.g., transcervically). For a more detailed description of one example of such an electrosurgical instrument, reference can be made to U.S. Patent Application Publication No. 2016/0089199 by Sartor et al., the entire contents of which are incorporated by reference herein. Although shown and described herein as an electrosurgical instrument, surgical instrument 10 can be any suitable surgical instrument configured to transect and/or resect tissue (e.g., scalpel).

Cutter 12 of surgical instrument 10 is then positioned in the uterine cavity “C” next to a first side of the fundus “F” adjacent to the tubal ostia “TO” of first one of the fallopian tubes “FT” where a cut line “CL” is identified in the fundus “F.” The clinician then cuts partially into the fundus “F” along the cut line “CL” with cutter 12 of surgical instrument 10 to define a flap “X” by the cut line “CL” that is configured to totally occlude tubal ostia “TO” of the adjacent fallopian tube “FT.” The clinician may then move the cutter 12 to a second side of the fundus “F” adjacent to the tubal ostia “TO” of the other fallopian tube “FT” (e.g., a second one) to form another flap “X” in the same manner with respect to the tubal ostia “TO” of this second fallopian tube “FT.”

Each cut line “CL” is positioned to enable the respective flaps “X” to depend from the fundus “F” (FIG. 3) and occlude the respective tubal ostia “TO” upon completion of the respective cuts. With the flaps “X” blocking off access into the passageway “P” of the respective fallopian tubes “FT”, free ends of the flaps “X” can be secured to the body “B” of the uterus “U.” For example, a fastener applier 20, such as a tack applier, can be inserted into the uterine cavity “C” to fire a fastener 22 (e.g., a tack) into each flap “X” to secure each flap “X” to the body “B” of the uterus “U.” As can be appreciated, any suitable fastener applier or stitching device (e.g., suture) can be utilized to secure flap “X” to the body “B” of the uterus “U.” For a more detailed description of such devices, reference can be made, for example, to U.S. Pat. No. 10,085,746 to Fischvogt et al., U.S. Pat. No. 9,358,010 to Wenchell et al., and/or U.S. Pat. No. 8,337,515 to Sniffin et al., the entire contents of each of which are incorporated by reference herein.

In some aspects of this disclosure, the flap “X” on the first side can be secured to the body “B” of the uterus “U” before the other flap “X” is formed.

Advantageously, once the flaps “X” are secured to the body “B” of the uterus “U,” the female patient is immediately sterile without any abdominal incisions or external scarring caused by such abdominal incisions. Over time, the flaps “X” will eventually adhere or otherwise become unitary with the body “B” of the uterus “U,” for example, through the natural build-up of scar tissue so that sterilization remains permanent, regardless of whether the fasteners absorb into the body.

Any of the disclosed fasteners (e.g., tacks, staples, clips, suture, etc.) may be bioabsorbable.

The various aspects disclosed herein may also be provided in connection with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effected by one skilled in the art without departing from the scope or spirit of this disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A method of sterilizing a female patient, the method comprising:
cutting into a fundus of a uterus of the female patient to form a flap; and
securing the flap to a wall of the uterus to occlude a fallopian tube of the female patient.

2. The method of claim 1, further comprising forming the flap adjacent to a tubal ostia of the fallopian tube.

3. The method of claim 2, further comprising forming a second flap from the fundus of the uterus.

4. The method of claim 3, further comprising positioning the second flap adjacent to a tubal ostia of a second fallopian tube of the female patient.

5. The method of claim 4, further comprising securing the second flap to the wall of the uterus to occlude the second fallopian tube.

6. The method of claim 1, wherein securing the flap includes fastening the flap to the body with a tack.

7. The method of claim 5, wherein securing the second flap includes fastening the second flap to the body with a tack.

8. The method of claim 1, further comprising advancing a cutter into the uterus transcervically.

9. The method of claim 8, wherein cutting into the fundus includes conducting electrical energy through the cutter.

10. The method of claim 1, wherein forming the flap includes positioning the flap to depend from the fundus.
